(19) United States
(12) Patent Application Publication
(10) Pub. No.: US 2010/0152139 A1
(43) Pub. Date: Jun. 17, 2010

(54) EMULSIFIED SKIN EXTERNAL PREPARATION AND METHOD FOR STABILIZING THE SKIN EXTERNAL PREPARATION

(75) Inventors: Tadashi Yoneda, Chiba-shi (JP); Naoko Ito, Chiba-shi (JP); Kazuo Furuya, Chiba-shijp (JP)

Correspondence Address: SUGHRUE MION, PLLC, 2100 PENNSYLVANIA AVENUE, N.W., SUITE 800, WASHINGTON, DC, 20037, US

(73) Assignee: SHOWA DENKO K.K., Tokyo (JP)

(21) Appl. No.: 11/996,887

(22) PCT Filed: Jul. 24, 2006
(86) PCT No.: PCT/JP2006/315067

§ 371 Date: Jan. 25, 2008

Related U.S. Application Data
(60) Provisional application No. 60/704,879, filed on Aug. 3, 2005.

(30) Foreign Application Priority Data
Jul. 27, 2005 (JP) 2005-217645

(51) Int. Cl. A61K 8/55; A61K 8/67; A61Q 19/00

(52) U.S. Cl. 514/99

(57) ABSTRACT

An emulsified skin external preparation containing a salt of a higher fatty acid ester of ascorbic acid-2-phosphate is provided, and the decomposition and consequent decrease of the salt are prevented and thereby the stability of the preparation is enhanced. A method for stabilizing the skin external preparation containing a salt of a higher fatty acid ester of ascorbic acid-2-phosphate is provided. The emulsified skin external preparation includes 0.03 to 25% by mass of a salt of a higher fatty acid ester of ascorbic acid-2-phosphate (A), and 0.05 to 25% by mass of a glycerin mono fatty acid ester (B), the higher fatty acid ester of ascorbic acid-2-phosphate being represented by Formula (1): . . . (1) wherein R is a higher fatty acid residue.

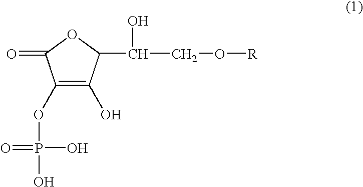

### CROSS REFERENCES OF RELATED APPLICATION

This application is an application filed under 35 U.S.C. §111 (a) claiming benefit pursuant to 35 U.S.C. §119(e) of the filing date of Provisional Application 60/704,879 filed on Aug. 3, 2005, pursuant to 35 U.S.C. §111 (b).

### TECHNICAL FIELD

The present invention relates to a stable emulsified skin external preparation containing a salt of a higher fatty acid ester of ascorbic acid-2-phosphate, and a glycerin mono fatty acid ester.

The invention also relates to a method for stabilizing the emulsified skin external preparation containing a salt of a higher fatty acid ester of ascorbic acid-2-phosphate.

### BACKGROUND ART

Ascorbic acid and derivatives thereof are known to produce effects such as skin whitening, antioxidation and stimulation of collagen synthesis, and are found in medicines, cosmetics and diets.

Ascorbic acid derivatives derived by esterifying the hydroxyl group at the 2-position with phosphoric acid and esterifying the hydroxyl group at the 6-position with a higher fatty acid, and salts of the derivatives are resistant to oxidation and are stable. They are amphiphilic and are consequently highly biocompatible, quickly penetrating in body tissues such as skin. They are expected to be used in medicines, cosmetics and diets. However, formulating such compounds and salts into skin external preparations has been unable to produce sufficient effects due to decomposition of the compounds and salts in the preparations.

To solve such problems, JP-A-H08-113525 discloses skin whitening skin external preparations wherein ascorbic acids and higher fatty acid esters of ascorbic acids are included in cyclodextrin to obtain higher stability, and polyhydric alcohols are used as solvents.

JP-A-2002-348228 discloses dermatological compositions wherein ascorbic acids, ester derivatives thereof, ether derivatives thereof or salts thereof are stabilized in an aqueous medium including water and glycol ethers.

JP-A-2002-3330 discloses cosmetics including water-soluble ascorbic acid derivatives such as magnesium ascorbate phosphate, and water and 1,2-alkanediol. The cosmetics are described to be stable over time.

None of these documents describe emulsified skin external preparations containing salts of higher fatty acid esters of ascorbic acid-2-phosphate.

### DISCLOSURE OF INVENTION

It is an object of the present invention to provide an emulsified skin external preparation containing a salt of a higher fatty acid ester of ascorbic acid-2-phosphate, wherein the decomposition and consequent decrease of the salt are prevented and thereby the stability of the preparation is enhanced.

It is another object of the invention that the provision of the skin external preparation provides a method for stabilizing the skin external preparation containing a salt of a higher fatty acid ester of ascorbic acid-2-phosphate.

The present inventors studied diligently in view of the aforesaid problems and have found that the objects are achieved by adding glycerin mono fatty acid esters. The invention has been completed based on the finding.

The invention is concerned with the following.

[1] An emulsified skin external preparation comprising 0.03 to 25% by mass of a salt of a higher fatty acid ester of ascorbic acid-2-phosphate (A), and 0.05 to 25% by mass of a glycerin mono fatty acid ester (B), the higher fatty acid ester of ascorbic acid-2-phosphate being represented by Formula (1):

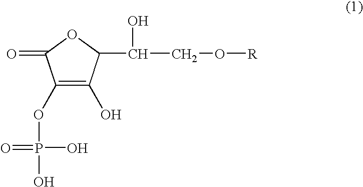

wherein R is a higher fatty acid residue.

[2] The emulsified skin external preparation as described in [1], wherein R in Formula (1) is a residue of an aliphatic carboxylic acid of 10 to 20 carbon atoms.

[3] The emulsified skin external preparation as described in [2], wherein the aliphatic carboxylic acid of 10 to 20 carbon atoms is lauric acid, myristic acid, palmitic acid, stearic acid, 2-hexyldecanoic acid or isostearic acid.

[4] The emulsified skin external preparation as described in [2], wherein the aliphatic carboxylic acid of 10 to 20 carbon atoms is palmitic acid.

[5] The emulsified skin external preparation as described in [2], wherein the aliphatic carboxylic acid of 10 to 20 carbon atoms is 2-hexyldecanoic acid.

[6] The emulsified skin external preparation as described in any one of [1] to [5], wherein the component (A) is a sodium salt.

[7] The emulsified skin external preparation as described in [1], wherein the glycerin mono fatty acid ester (B) is at least one ester selected from the group consisting of glyceryl caprylate, glyceryl caprate, glyceryl laurate, glyceryl myristate, glyceryl stearate, glyceryl isostearate, glyceryl behenate, glyceryl linoleate, glyceryl oleate, glyceryl cocofatty acid ester, glyceryl ricinoleate, glyceryl hydroxystearate and glyceryl erucate.

[8] An emulsified cosmetic comprising the emulsified skin external preparation of any one of [1] to [7].

[9] A method of stabilizing an emulsified skin external preparation, characterized in that a salt of a higher fatty acid ester of ascorbic acid-2-phosphate (A) is used in combination with a glycerin mono fatty acid ester (B), the higher fatty acid ester of ascorbic acid-2-phosphate being represented by Formula (1):

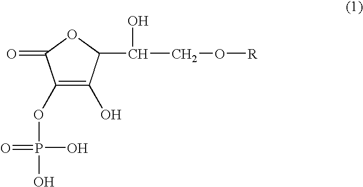

wherein R is a higher fatty acid residue.

The emulsified skin external preparation according to the invention has superior stability because the decomposition and consequent decrease of the salt of the higher fatty acid ester of ascorbic acid-2-phosphate are prevented. The emulsified skin external preparation is useful as general skin external agents, particularly useful in cosmetics.

### BEST MODE FOR CARRYING OUT THE INVENTION

The emulsified skin external preparation and stabilizing method thereof according to the present invention will be described in detail below.

### <Emulsified Skin External Preparation>

The emulsified skin external preparation of the invention includes a salt of a higher fatty acid ester of ascorbic acid-2-phosphate (A), and a glycerin mono fatty acid ester (B).

(A) Salt of Higher Fatty Acid Ester of Ascorbic acid-2-phosphate

The salt (A) is based on a higher fatty acid ester of ascorbic acid-2-phosphate (hereinafter, ascorbic acid ester) represented by Formula (1):

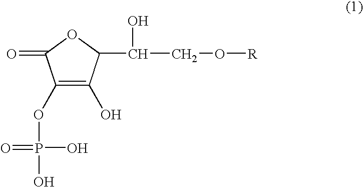

wherein R is a higher fatty acid residue. Preferred examples of the higher fatty acids include aliphatic carboxylic acids of 10 to 20 carbon atoms, such as lauric acid, myristic acid, palmitic acid, stearic acid, 2-hexyldecanoic acid and isostearic acid. Of these, palmitic acid and 2-hexyldecanoic acid are more preferable.

The salts of ascorbic acid ester (A) are compounds in which the phosphoric acid residue involved in the phosphate bond in the ascorbic acid ester forms a salt and/or compounds in which the hydroxyl group at the 3-position of the ascorbic acid forms a salt with a base. Examples of the salts of ascorbic acid ester include sodium salt, potassium salt, calcium salt, magnesium salt and zinc salt, with the sodium salt being preferred.

The salts of ascorbic acid ester (A) may be used singly or in combination of two or more kinds. The salts of ascorbic acid ester (A) generally account for 0.03 to 25% by mass, preferably 0.1 to 10% by mass, more preferably 0.5 to 5% by mass of the skin external preparation. When the amount of the salts (A) is less than the above range, the skin external preparation often does not produce desired effects. Any amount exceeding the above range is not effective because further improvements in effects are not expected.

### (B) Glycerin Mono Fatty Acid Ester

The glycerin mono fatty acid ester (B) used in the invention is a compound in which one molecule of a fatty acid is ester bonded to any hydroxyl group of glycerin. The ester is otherwise called monoacylglycerol or monoglyceride. Examples of the fatty acids include straight-chain fatty acids, branched-chain fatty acids, saturated fatty acids, unsaturated fatty acids and hydroxy fatty acids.

The addition of the glycerin mono fatty acid ester (B) prevents the decomposition and consequent decrease of the salt of the higher fatty acid ester of ascorbic acid-2-phosphate in the preparation, and thereby improves the stability of the preparation.

Examples of the glycerin mono fatty acid esters (B) include glyceryl caprylate, glyceryl caprate, glyceryl laurate, glyceryl myristate, glyceryl stearate, glyceryl linoleate, glyceryl oleate, glyceryl isostearate, glyceryl behenate, glyceryl erucate, glyceryl cocofatty acid ester, glyceryl ricinoleate, glyceryl hydroxystearate, wheat germ oil fatty acid monoglyceride, safflower oil fatty acid monoglyceride, hydrogenated soybean fatty acid monoglyceride, saturated fatty acid monoglyceride, cotton seed oil fatty acid monoglyceride, tallow fatty acid monoglyceride and lanolin fatty acid monoglyceride.

Of these, glyceryl caprylate, glyceryl caprate, glyceryl laurate, glyceryl myristate, glyceryl stearate, glyceryl linoleate, glyceryl oleate, glyceryl isostearate, glyceryl behenate, glyceryl erucate, glyceryl cocofatty acid ester, glyceryl ricinoleate and glyceryl hydroxystearate are preferred, and glyceryl myristate, glyceryl stearate and glyceryl isostearate are particularly preferred.

The glycerin mono fatty acid esters (B) may be used singly or in combination of two or more kinds. The glycerin mono fatty acid esters (B) generally account for 0.05 to 25% by mass, preferably 0.5 to 10% by mass, more preferably 1 to 5% by mass of the skin external preparation. This amount of the components (B) enables the stable skin external preparation.

### (C) Other Components

The skin external preparation may contain a component (C) that is commonly used in the skin external preparations, together with the salt of ascorbic acid ester (A) and the glycerin mono fatty acid ester (B). Examples of the components (C) include:

hydrocarbons such as ozokerite, α-olefin oligomers, light isoparaffin, light liquid isoparaffin, squalene, squalane, synthetic squalane, vegetable squalane, ceresin, paraffin, polyethylene powder, polybutene, microcrystalline wax, liquid isoparaffin, liquid paraffin, mineral oil and vaseline;

natural fats and oils, such as natural waxes including jojoba oil, carnauba wax, candelilla wax, rice bran wax, shellac, lanolin, mink oil wax, whale wax, sugarcane wax, sperm oil, beeswax and montan wax; avocado oil, almond oil, olive oil, extra virgin olive oil, sesame oil, rice bran oil, rice oil, rice germ oil, corn oil, safflower oil, soybean oil, maize oil, rapeseed oil, persic oil, palm kernel oil, palm oil, castor oil, sunflower oil, high oleic sunflower oil, grape seed oil, cotton seed oil, coconut oil, hydrogenated coconut oil, beef tallow, hydrogenated oil, horse oil, mink oil, egg yolk oil, egg yolk fatty oil, rose hip oil, kukui nut oil, evening primrose oil, wheat germ oil, peanut oil, camellia oil, sasanqua oil, cacao butter, Japanese wax, beef bone fat, neatsfoot oil, lard, horse fat, mutton tallow, shea butter, macadamia nut oil and meadowfoam oil;

fatty acids such as lauric acid, myristic acid, palmitic acid, stearic acid, behenic acid, oleic acid, linolic acid, linolenic acid, γ-linolenic acid, isostearic acid, 12-hydroxystearic acid, undecylenic acid and coconut fatty acid;

higher alcohols such as isostearyl alcohol, octyldodecanol, hexyldecanol, cholesterol, phytosterol, lauryl alcohol, myristyl alcohol, cetanol, stearyl alcohol, oleyl alcohol, behenyl alcohol and cetostearyl alcohol;

alkyl glyceryl ethers such as batyl alcohol, chimyl alcohol, selachyl alcohol and isostearyl glyceryl ether;

esters such as isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol di(caprylate caprate), propylene glycol dicaprate, propylene glycol dioleate, neopentyl glycol dicaprate, neopentyl glycol dioctanoate, glyceryl tricaprylate, glyceryl tri-2-ethylhexanoate, glyceryl tri(caprylate caprate), glyceryl tri(caprylate caprate stearate), glyceryl triundecylate, glyceryl triisopalmitate, glyceryl triisostearate, trimethylolpropane tri-2-ethylhexanoate, trimethylolpropane triisostearate, pentaerythrityl tetra-2-ethylhexanoate, pentaerythrityl tetramyristate, pentaerythrityl tetraisostearate, diglyceryl tetraisostearate, octyldodecyl neopentanoate, isocetyl octanoate, isostearyl octanoate, 2-ethylhexyl isopelargonate, hexyldecyl dimethyloctanoate, octyldodecyl dimethyloctanoate, 2-ethylhexyl isopalmitate, isocetyl isostearate, isostearyl isostearate, octyldodecyl isostearate, lauryl lactate, myristyl lactate, cetyl lactate, octyldodecyl lactate, triethyl citrate, acetyltriethyl citrate, acetyltributyl citrate, trioctyl citrate, triisocetyl citrate, trioctyldodecyl citrate, diisostearyl malate, 2-ethylhexyl hydroxystearate, di-2-ethylhexyl succinate, diisopropyl adipate, diisobutyl adipate, dioctyl adipate, diheptylundecyl adipate, diethyl sebacate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoylhydroxystearate, stearyl 12-stearoylhydroxystearate, isostearyl 12-stearoylhydroxystearate, polyoxyethylene (3) polyoxypropylene (1) cetyl ether acetate, polyoxyethylene (3) polyoxypropylene (1) isocetyl ether acetate, isononyl isononanoate, octyl isononanoate, tridecyl isononanoate and isotridecyl isononanoate;

silicone oils such as methyl polysiloxane, methylphenyl polysiloxane, methylhydrogen polysiloxane, methyl cyclopolysiloxane, octamethyl cyclotetrasiloxane, decamethyl cyclopentasiloxane, dodecamethyl cyclohexasiloxane, octamethyl trisiloxane, decamethyl tetrasiloxane, tetradecamethyl hexasiloxane, highly polymerized methyl polysiloxane, dimethyl siloxane/methyl(polyoxyethylene)siloxane/methyl(polyoxypropylene)siloxane copolymer, dimethyl siloxane/methyl(polyoxyethylene)siloxane copolymer, dimethyl siloxane/methyl(polyoxypropylene)siloxane copolymer, dimethyl siloxane/methyl cetyloxysiloxane copolymer, dimethyl siloxane/methyl stearoxysiloxane copolymer, polyether-modified silicones, alcohol-modified silicones, alkyl-modified silicones and amino-modified silicones;

polymers such as sodium alginate, carrageenan, agar, furcelleran, cyamoposis gum, pyrus cydonia seed, konjac mannan, tamarind gum, tara gum, dextrin, starch, locust bean gum, gum arabic, ghatti gum, karaya gum, tragacanth gum, arabinogalactan, pectin, marmelo, chitosan, starch, curdlan, xanthan gum, gellan gum, cyclodextrin, dextran, pullulan, microcrystalline cellulose, methylcellulose, ethylcellulose, hydroxyethylcellulose, hydroxypropylcellulose, hydroxypropylmethylcellulose, carboxymethylcellulose, carboxy starch, cationized cellulose, starch phosphate, cationized cyamoposis gum, carboxymethyl/hydroxypropylated cyamoposis gum, hydroxypropylated cyamoposis gum, albumin, casein, gelatin, sodium polyacrylate, polyacrylic acid amide, carboxyvinyl polymers, polyethyleneimine, highly polymerized polyethylene glycol, polyvinyl alcohol, polyvinylpyrrolidone, polyvinyl ether, polyacrylamide, acrylic acid copolymers, methacrylic acid copolymers, maleic acid copolymers, vinylpyridine copolymers, ethylene/acrylic acid copolymers, vinylpyrrolidone polymers, vinyl alcohol/vinylpyrrolidone copolymers, nitrogen-substituted acrylamide polymers, amino-modified silicones, cationized polymers, dimethylacryl ammonium polymers, acrylic acid-based anionic polymers, methacrylic acid-based anionic polymers, modified silicones, alkyl (C₁₀₋₃₀) acrylate or methacrylate copolymers and polyoxyethylene/polyoxypropylene copolymer;

alcohols such as ethanol, isopropyl alcohol, 1-butanol, 2-butanol and benzyl alcohol;

dihydric alcohols such as ethylene glycol, propylene glycol, 1,3-butanediol, 3-methyl-1,3-butanediol, 1,2-pentanediol, 1,2-hexanediol, diethylene glycol, triethylene glycol, polyethylene glycol, dipropylene glycol and polypropylene glycol;

trihydric alcohols such as glycerol;

ethers of trihydric alcohols such as diglycerol and polyglycerol;

sugar alcohols such as mannitol, sorbitol, xylitol, maltitol, erythritol and pentaerythritol;

monosaccharides such as glucose, fructose and xylose;

oligosaccharides such as sucrose, lactose, maltose and trehalose;

anionic surfactants such as potassium coconut fatty acid ester, sodium coconut fatty acid ester, triethanolamine coconut fatty acid ester, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, sodium castor oil fatty acid ester, zinc undecylenate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate, polyoxyethylene laurylether acetic acid, sodium polyoxyethylene laurylether acetate, polyoxyethylene tridecylether acetic acid, sodium polyoxyethylene tridecylether acetate, sodium stearoyl lactate, sodium isostearoyl lactate, lauroylsarcosine sodium, sarcosine coconut fatty acid ester, sarcosine sodium coconut fatty acid ester, sarcosine triethanolamine coconut fatty acid ester, lauroyl sarcosine, lauroyl sarcosine potassium, lauroyl sarcosine triethanolamine, oleoyl sarcosine, myristoyl sarcosine sodium, sodium stearoyl glutamate, coconut fatty acid acylglutamic acid, potassium coconut fatty acid acylglutamate, sodium coconut fatty acid acylglutamate, triethanolamine coconut fatty acid acylglutamate, lauroyl acylglutamic acid, potassium lauroyl acylglutamate, sodium lauroyl acylglutamate, triethanolamine lauroyl acylglutamate, myristoyl acylglutamic acid, potassium myristoyl acylglutamate, sodium myristoyl acylglutamate, stearoyl acylglutamic acid, potassium stearoyl acylglutamate, disodium stearoyl acylglutamate, sodium hydrogenated tallow fatty acid acylglutamate, sodium coconut fatty acid/hydrogenated tallow fatty acid acylglutamate, methylalanine sodium coconut fatty acid ester, lauroyl methylalanine, lauroyl methylalanine sodium, lauroyl methylalanine triethanolamine, myristoyl methylalanine sodium, lauroyl methyltaurine sodium, methyltaurine potassium coconut fatty acid ester, methyltaurine sodium coconut fatty acid ester, methyltaurine magnesium coconut fatty acid ester, myristoyl methyltaurine sodium, palmitoyl methyltaurine sodium, stearoyl methyltaurine sodium, oleoyl methyltaurine sodium, sodium alkanesulfonate, sodium tetradecenesulfonate, dioctylsodium sulfosuccinate, lauryl disodium sulfosuccinate, ethyl coconut fatty acid ester sodium sulfonate, sodium laurylsulfate, triethanolamine laurylsulfate, sodium cetyl sulfate, triethanolamine alkylsulfates (11, 13, 15), sodium alkylsulfates (12, 13), triethanolamine alkylsulfates (12, 13), ammonium alkylsulfates (12, 14, 16), diethanolamine alkylsulfates (12, 13), triethanolamine alkylsulfates (12-14), triethanolamine alkylsulfates (12-15), magnesium triethanolamine cocoalkylsulfate, ammonium laurylsulfate, potassium laurylsulfate, magnesium laurylsulfate, monoethanolamine laurylsulfate, diethanolamine laurylsulfate, sodium myristylsulfate, sodium stearylsulfate, sodium oleylsulfate, triethanolamine oleylsulfate, sodium polyoxyethylene laurylether sulfate, triethanolamine polyoxyethylene laurylether sulfate, sodium polyoxyethylene (1) alkyl (11, 13, 15) ether sulfate, triethanolamine polyoxyethylene (1) alkyl (11, 13, 15) ether sulfate, sodium polyoxyethylene (3) alkyl (11-15) ether sulfate, sodium polyoxyethylene (2) alkyl (12, 13) ether sulfate, sodium polyoxyethylene (3) alkyl (12-14) ether sulfate, sodium polyoxyethylene (3) alkyl (12-15) ether sulfate, sodium polyoxyethylene (2) laurylether sulfate, sodium polyoxyethylene (3) myristylether sulfate, sodium higher fatty acid alkanolamide sulfate, laurylphosphoric acid, sodium laurylphosphate, potassium cetylphosphate, diethanolamine cetylphosphate, polyoxyethylene oleylether phosphoric acid, polyoxyethylene laurylether phosphoric acid, sodium polyoxyethylene laurylether phosphate, polyoxyethylene cetylether phosphoric acid, sodium polyoxyethylene cetylether phosphate, polyoxyethylene stearylether phosphoric acid, polyoxyethylene oleylether phosphoric acid, sodium polyoxyethylene oleylether phosphate, polyoxyethylene alkylphenyl ether phosphoric acid, sodium polyoxyethylene alkylphenyl ether phosphate, triethanolamine polyoxyethylene alkylphenyl ether phosphate, polyoxyethylene octylether phosphoric acid, polyoxyethylene (10) alkyl (12, 13) ether phosphoric acid, polyoxyethylene alkyl (12-15) ether phosphoric acid, polyoxyethylene alkyl (12-16) ether phosphoric acid, triethanolamine polyoxyethylene laurylether phosphate and diethanolamine polyoxyethylene oleylether phosphate;

cationic surfactants such as dioctylamine, dimethylstearylamine, trilaurylamind, stearic acid diethylaminoethylamide, lauryltrimethylammonium chloride, cetyltrimethylammonium chloride, cetyltrimethylammonium bromide, cetyltrimethylammonium saccharin, stearyltrimethylammonium chloride, alkyl (20-22) trimethylammonium chloride, lauryltrimethylammonium bromide, alkyl (16, 18) trimethylammonium chloride, stearyltrimethylammonium bromide, stearyltrimethylammonium saccharin, alkyl (28) trimethylammonium chloride, di(polyoxyethylene)oleylmethylammonium chloride (2EO), dipolyoxyethylenestearylmethylammonium chloride, polyoxyethylene (1) polyoxypropylene (25) diethylmethylammonium chloride, tri(polyoxyethylene)stearylammonium chloride (5EO), distearyldimethylammonium chloride, dialkyl (12-15) dimethylammonium chloride, dialkyl (12-18) dimethylammonium chloride, dialkyl (14-18) dimethylammonium chloride, dicocoyldimethylammonium chloride, dicetyldimethylammonium chloride, isostearyllauryldimethylammonium chloride, benzalkonium chloride, myristyldimethylbenzylammonium chloride, lauryldimethyl(ethylbenzyl)ammonium chloride, stearyldimethylbenzylammonium chloride, laurylpyridinium chloride, cetylpyridinium chloride, lauroylcolaminoformylmethylpyridinium chloride, stearoylcolaminoformylmethylpyridinium chloride, alkylisoquinolium bromide, methylbenzethonium chloride and benzethonium chloride;

amphoteric surfactants such as 2-alkyl-N-carboxymethyl-N-hydroxyethyl imidazolinium betaine, alkyldiaminoethylglycine hydrochloride, lauryldiaminoethylglycine sodium, undecylhydroxyethylimidazolium betaine sodium, undecyl-N-carboxymethylimidazolium betaine, acyl-N-carboxyethyl-N-hydroxyethylethylenediamine disodium coconut fatty acid ester, acyl-N-carboxyethoxyethyl-N-carboxyethylethylenediamine disodium coconut fatty acid ester, acyl-N-carboxymethoxyethyl-N-carboxymethylethylenediamine disodium coconut fatty acid ester, sodium laurylaminopropionate, sodium laurylaminodipropionate, triethanolamine laurylaminopropionate, acyl-N-carboxyethyl-N=hydroxyethylethylenediamine sodium palm oil fatty acid ester, betaine lauryldimethylaminoacetate, betaine coconut oil alkyldimethylaminoacetate, betaine stearyldimethylaminoacetate, stearyldimethyl betaine sodium, amidopropylbetaine coconut fatty acid ester, amidopropylbetaine palm oil fatty acid ester, lauric acid amide betaine propylacetate, amidopropylbetaine ricinoleate, stearyldihydroxyethyl betaine and laurylhydroxysulfobetaine;

nonionic surfactants such as polyoxyethylene (10) alkyl (12, 13) ether, polyoxyethylene lauryl ether, polyoxyethylene cetyl ether, polyoxyethylene stearyl ether, polyoxyethylene oleyl ether, polyoxyethylene (3, 7, 12) alkyl (12-14) ether, polyoxyethylene tridecyl ether, polyoxyethylene myristyl ether, polyoxyethylene-sec-alkyl (14) ether, polyoxyethylene isocetyl ether, polyoxyethylene cetostearyl ether, polyoxyethylene (2, 10, 20) isostearyl ether, polyoxyethylene oleylcetyl ether, polyoxyethylene (20) aralkyl ether, polyoxyethylene octyldodecyl ether, polyoxyethylene behenyl ether, polyoxyethylene octylphenyl ether, polyoxyethylene nonylphenyl ether, polyoxyethylene dinonylphenyl ether, polyoxyethylene (1) polyoxypropylene (1, 2, 4, 8) cetyl ether, polyoxyethylene (5) polyoxypropylene (1, 2, 4, 8) cetyl ether, polyoxyethylene (10) polyoxypropylene (1, 2, 4, 8) cetyl ether, polyoxyethylene (20) polyoxypropylene (1, 2, 4, 8) cetyl ether, polyoxyethylene polyoxypropylene lauryl ether, polyoxyethylene (3) polyoxypropylene (34) stearyl ether, polyoxyethylene (4) polyoxypropylene (30) stearyl ether, polyoxyethylene (34) polyoxypropylene (23) stearyl ether, polyoxyethylene polyoxypropylene cetyl ether, polyoxyethylene polyoxypropylene decyltetradecyl ether, polyethylene glycol monolaurate, ethylene glycol monostearate, polyethylene glycol monostearate, polyethylene glycol monooleate, ethylene glycol fatty acid ester, self-emulsifiable ethylene glycol monostearate, diethylene glycol laurate, polyethylene glycol myristate, polyethylene glycol palmitate, diethylene glycol stearate, self-emulsifiable polyethylene glycol (2) monostearate, polyethylene glycol isostearate, ethylene glycol dioctanoate, diethylene glycol dilaurate, polyethylene glycol dilaurate, polyethylene glycol (150) dipalmitate, ethylene glycol distearate, diethylene glycol distearate, polyethylene glycol distearate, ethylene glycol dioleate, polyethylene glycol dioleate, polyethylene glycol diricinoleate, polyoxyethylene (20) sorbitan monolaurate, polyoxyethylene (20) sorbitan monopalmitate, polyoxyethylene (6) sorbitan monostearate, polyoxyethylene (20) sorbitan monostearate, polyoxyethylene (20) sorbitan tristearate, polyoxyethylene (6) sorbitan monooleate, polyoxyethylene (20) sorbitan monooleate, polyoxyethylene (20) sorbitan trioleate, sorbitan polyoxyethylene (20) coconut fatty acid ester, polyoxyethylene (10-80) sorbitan monolaurate, polyoxyethylene sorbitan tristearate, polyoxyethylene (20) sorbitan isostearate, polyoxyethylene (150) sorbitan tristearate, polyoxyethylene castor oil, polyoxyethylene hydrogenated castor oil, polyoxyethylene (10) hydrogenated castor oil, polyoxyethylene (20) hydrogenated castor oil, polyoxyethylene (40) hydrogenated castor oil, polyoxyethylene (50) hydrogenated castor oil, polyoxyethylene (60) hydrogenated castor oil, glyceryl monoisostearate monomyristate, glyceryl sesquioleate, glyceryl distearate, glyceryl diisostearate, glyceryl diarachidate, sorbitan monolaurate, sorbitan monopalmitate, sorbitan monostearate, sorbitan monoisostearate, sorbitan monooleate, sorbitan sesquistearate, sorbitan sesquioleate, sorbitan tristearate, sorbitan trioleate, sorbitan coconut fatty acid ester, sorbitan isostearate, sorbitan sesquiisostearate, sorbitan distearate, diglyceryl isopalmitate, poly (4-10) glyceryl monolaurate, poly (10) glyceryl monomyristate, poly (2-10) glyceryl monostearate, poly (2-10) glyceryl monoisostearate, poly (2-10) glyceryl monooleate, diglyceryl sesquioleate, poly (2-10) glyceryl diisostearate, poly (6-10) glyceryl distearate, diglyceryl triisostearate, poly (10) glyceryl tristearate, poly (10) glyceryl trioleate, poly (2) glyceryl tetraisostearate, decaglyceryl pentastearate, poly (6-10) glyceryl pentaoleate, poly (10) glyceryl heptastearate, decaglyceryl decastearate, poly (10) glyceryl decaoleate, condensed poly (6) glyceryl ricinoleate, sucrose fatty acid ester, sucrose coconut fatty acid ester, alkyl glucoside, coconut oil alkyldimethylamine oxide, lauryldimethylamine oxide, dihydroxyethyllauryldimethylamine oxide, stearyldimethylamine oxide, oleyldimethylamine oxide and polyoxyethylene coconut oil alkyldimethylamine oxide;

natural surfactants such as saponin, lecithin, soybean phospholipid, hydrogenated soybean phospholipid, soybean lysophospholipid, hydrogenated soybean lysophospholipid, egg yolk lecithin, hydrogenated egg yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophoroliquid and mannosylerythritol lipid;

ultraviolet light absorbers, including paraminobenzoic acid derivatives such as paraminobenzoic acid, ethyl paraminobenzoate, glyceryl paraminobenzoate, amyl paradimethylaminobenzoate and 2-ethylhexyl paradimethylaminobenzoate, cinnamic acid derivatives such as benzyl cinnamate, glyceryl diparamethoxycinnamate mono-2-ethylhexanoate, methyl 2,4-diisopropylcinnamate, ethyl 2,4-diisopropylcinnamate, potassium paramethoxycinnamate, sodium paramethoxycinnamate, isopropyl paramethoxycinnamate, 2-ethylhexyl paramethoxycinnamate, 2-ethoxyethyl paramethoxycinnamate and ethyl paraethoxycinnamate, urocanic acid derivatives such as urocanic acid and ethyl urocanate, benzophenone derivatives such as 2,4-dihydroxybenzophenone, 2,2′,4,4′-tetrahydroxybenzophenone, 2-hydroxy-4-methoxy-5-sulfobenzophenonesodium, 2-hydroxy-4-methoxybenzophenone-5-sulfonic acid, 2-hydroxy-4-methoxybenzophenone, 2,2′-dihydroxy-4,4′-dimethoxybenzophenone and 2,2′-dihydroxy-4,4′-dimethoxy-5-sulfobenzophenonesodium, salicylic acid derivatives such as ethylene glycol salicylate, 2-ethylhexyl salicylate, phenyl salicylate, benzyl salicylate, p-tert-butylphenyl salicylate, homomethyl salicylate and 3,3,5-trimethylcyclohexyl salicylate, 2-(2′-hydroxy-5′-methoxyphenyl)benzotriazole and 4-tert-butyl-4′-methoxybenzoylmethane;

powders and color materials such as kaolin, silicic anhydride, aluminum magnesium silicate, sericite, talc, boron nitride, mica, montmorillonite, hemp cellulose powder, wheat starch, silk powder, cornstarch, natural dyes including nitro dye, azo dye, nitroso dye, triphenylmethane dye, xanthene dye, quinoline dye, anthraquinone dye, indigo dye, pyrene dye, phthalocyanine dye, flavonoid, quinone, porphyrin, water-soluble annatto, squid ink powder, caramel, guaiazulene, gardenia blue, gardenia yellow, cochineal, shikonin, copper chlorophyllin sodium, paprika dye, safflower red, safflower yellow, laccaic acid and riboflavin butyrate, carbon black, yellow iron oxide, black iron oxide, red iron oxide, iron blue, ultramarine blue, zinc oxide, chromium oxide, titanium oxide, black titanium oxide, zirconium oxide, chromium hydroxide, alumina, magnesium oxide, barium sulfate, aluminum hydroxide, calcium carbonate, lithium cobalt titanate, manganese violet and pearl pigment;

plant extracts such as angelica keiskei extract, gambir extract, avocado extract, hydrangea serrata leaf extract, gynostemma pentaphyllum extract, althea extract, arnica extract, oil-soluble arnica extract, almond extract, aloe extract, styrax benzoin resin extract, ginkgo extract, urtica extract, orris root extract, fennel extract, turmeric extract, rose fruit extract, echinacea leaf extract, scutellaria baicalensis root extract, phellodendron bark extract, coptis rhizome extract, hordeum vulgare seed extract, gumbo extract, hypericum erectum extract, oil-soluble hypericum erectum extract, lamium album flower extract, oil-soluble lamium album flower extract, ononis extract, nasturtium officinale extract, orange extract, orange flower water, seaweed extract, kaki tannin, puerariae radix extract, valerian extract, cattail extract, chamomilla extract, oil-soluble chamomilla extract, chamomilla water, oat extract, carrot extract, oil-soluble carrot extract, carrot oil, artemisia capillaris extract, licorice extract, licorice extract powder, licorice flavonoid, cantharis tincture, raspberry extract, kiwi extract, cinchona bark extract, cucumber extract, apricot kernel extract, quince seed extract, gardenia extract, sasa veitchii extract, sophora angustifolia extract, walnut shell extract, grapefruit extract, clematis extract, brown sugar extract, chlorella extract, mulberry extract, cinnamon bark extract, gentian extract, geranium herb extract, tea extract, spatterdock extract, arctium lappa root extract, oil-soluble arctium lappa root extract, wheat germ extract, hydrolyzed wheat powder, rice bran extract, rice bran fermentation extract, comfrey extract, asiasarum root extract, saffron extract, saponaria officinalis extract, oil-soluble salvia extract, crataegus cuneata fruit extract, xanthoxylum extract, shiitake mushroom extract, shiitake mushroom extract powder, rehmannia glutinosa extract, sycon extract, oil-soluble sycon extract, Japanese basil extract, linden extract, oil-soluble linden extract, filipendula multijuga extract, crude drug extract, coix lacryma-jobi seed extract, ginger extract, oil-soluble ginger extract, ginger tincture, acorus calamus root extract, betula alba extract, oil-soluble betula alba extract, betula alba sap, lonicera extract, equisetum arvense extract, oil-soluble equisetum arvense extract, scordinin, stevia extract, ivy extract, crataegus oxyacantha extract, sambucus nigra flower extract, juniperus communis extract, achillea millefolium extract, oil-soluble achillea millefolium extract, mentha piperita extract, sage extract, oil-soluble sage extract, sage water, malva sylvestris extract, celery extract, cnidium officinale extract, cnidium officinale water, swertia japonica extract, soybean extract, jujube extract, thyme extract, camellia sinensis leaf extract, camellia sinensis dry distillate, camellia sinensis seed extract, clove flower extract, citrus unshiu peel extract, camellia japonica seed extract, centella asiatica extract, oil-soluble juglans regia extract, duke extract, terminalia extract, red pepper tincture, angelica acutiloba extract, oil-soluble angelica acutiloba extract, angelica acutiloba water, calendula officinalis flower extract, oil-soluble calendula officinalis flower extract, soymilk powder, prunus persica extract, citrus aurantium amara extract, houttuynia cordata extract, tomato extract, potentilla erecta root extract, natto extract, ginseng extract, oil-soluble ginseng extract, garlic extract, rosa canina fruit extract, oil-soluble rosa canina fruit extract, malt extract, malt root extract, ophiopogon tuber extract, parsley extract, hordeum vulgare leaf juice concentrate, distilled peppermint water, hamamelis water, hamamelis extract, rosa centifolia flower extract, parietaria extract, isodonis japonicus extract, eriobotrya japonica leaf extract, oil-soluble eriobotrya japonica leaf extract, coltsfoot flower extract, poria cocos extract, ruscus aculeatus root extract, ruscus aculeatus root extract powder, grape extract, grape leaf extract, grape water, hayflower extract, luffa cylindrica fruit extract, luffa cylindrica fruit water, safflower extract, oil-soluble tilia miqueliana extract, tilia miqueliana water, paeonia suffruticosa root extract, hops extract, oil-soluble hops extract, pinus sylvestris cone extract, silybum marianum fruit extract, horse chestnut extract, oil-soluble horse chestnut extract, sapindus mukurossi peel extract, melissa officinalis leaf extract, melilotus officinalis extract, peach leaf extract, oil-soluble peach leaf extract, bean-sprouts extract, centaurea cyanus flower extract, centaurea cyanus flower water, eucalyptus extract, saxifraga sarmentosa extract, lilium candidum bulb extract, coix lacryma jobi seed extract, oil-soluble coix lacryma jobi seed extract, artemisia princeps extract, artemisia princeps water, lavender extract, lavender water, apple extract, ganoderma lucidum extract, lettuce extract, lemon extract, astragalus sinicus extract, rose water, rosemary extract, oil-soluble rosemary extract, anthemis nobilis flower extract and sanguisorba officinalis root extract;

amino acids and peptides such as glycine, alanine, valine, leucine, isoleucine, serine, threonine, phenylalanine, tyrosine, tryptophan, cystine, cysteine, methionine, proline, hydroxyproline, aspartic acid, asparagine, glutamic acid, glutamine, arginine, histidine, lysine, γ-aminobutyric acid, DL-pyrrolidonecarboxylic acid, ε-aminocaproic acid, hydrolyzed elastin, water-soluble elastin, hydrolyzed collagen, water-soluble collagen, casein, glutathione, wheat peptide and soybean peptide;

vitamins and vitamin affecters, including vitamin A such as retinol, retinal, retinoic acid, retinol acetate and retinol palmitate,

carotenoids such as α-carotene, β-carotene, γ-carotene, δ-carotene, lycopene, zeaxanthin, cryptoxanthin, echinenone and astaxanthin,
vitamin B1 such as thiamines,
vitamin B2 such as riboflavin,
vitamin B6 such as pyridoxine, pyridoxal and pyridoxamine,
vitamin B12 such as cyanocobalamin,
folic acids, nicotinic acid, nicotinic acid amide, pantothenic acids, biotins,
vitamin C such as L-ascorbic acid, sodium L-ascorbate, L-ascorbyl stearate, L-ascorbyl palmitate, L-ascorbyl dipalmitate, L-ascorbyl tetraisopalmitate, disodium L-ascorbate sulfate, magnesium L-ascorbate, L-ascorbyl sodium phosphate and L-ascorbic acid-2-glucoside,
vitamin D such as ergocalciferol and cholecalciferol,
vitamin E such as d-α-tocopherol, DL-α-tocopherol, dl-α-tocopherol acetate, dl-α-tocopherol succinate, β-tocopherol, γ-tocopherol and d-δ-tocopherol, ubiquinones, vitamin K, carnitine, ferulic acid, γ-oryzanol, α-lipoic acid and orotic acid;

antiseptics such as benzoic acid, sodium benzoate, undecylenic acid, salicylic acid, sorbic acid, potassium sorbate, dehydroacetic acid, sodium dehydroacetate, isobutyl parahydroxybenzoate, isopropyl parahydroxybenzoate, ethyl parahydroxybenzoate, butyl parahydroxybenzoate, propyl parahydroxybenzoate, benzyl parahydroxybenzoate, methyl parahydroxybenzoate, methyl sodium parahydroxybenzoate, phenoxyethanol, photosensitive agent No. 101, photosensitive agent No. 201 and photosensitive agent No. 401;

antioxidants such as butylhydroxyanisole, butylhydroxytoluene, propyl gallate, erythorbic acid, sodium erythorbate, parahydroxyanisole and octyl gallate;

sequestering agents such as trisodium ethylenediaminehydroxyethyltriacetate, edetic acid, disodium edetate, trisodium edetate, tetrasodium edetate, sodium citrate, gluconic acid, phytic acid, sodium polyphosphate and sodium metaphosphate;

moisturizers such as hyaluronic acid, sodium hyaluronate, sodium chondroitinsulfate, sodium lactate, sodium pyrrolidonecarboxylate, betaine, lactic acid bacteria culture solution, yeast extract and ceramide;

anti-inflammatory agents such as glycyrrhizinic acid, trisodium glycyrrhizinate, dipotassium glycyrrhizinate, monoammonium glycyrrhizinate, β-glycyrrhetinic acid, glyceryl glycyrrhetinate, stearyl glycyrrhetinate, lysozyme chloride, hydrocortisone and allantoin;

pH adjusters such as sodium hydroxide, potassium hydroxide and triethanolamine;

salts such as sodium chloride, potassium chloride, magnesium chloride and sodium sulfate;

α-hydroxy acids such as citric acid, glycolic acid, tartaric acid and lactic acid;

whitening agents such as arbutin, α-arbutin and placental extract;

essential oils such as angelica oil, ylang ylang oil, elemi oil, orange oil, German chamomile oil, anthemis nobilis oil, cardamom oil, calamus oil, galbanum oil, camphor oil, carrot seed oil, clary sage oil, grapefruit oil, clove oil, cinnamon bark oil, coriander oil, cypress oil, sandalwood oil, cedarwood oil, citronella oil, cinnamon leaf oil, jasmine absolute, juniper berry oil, ginger extract, spearmint oil, sage oil, cedar oil, geranium oil, thyme oil, tea tree oil, nutmeg oil, niaouli oil, neroli oil, pine oil, basil oil, peppermint oil, patchouli oil, palmarosa oil, fennel oil, petitgrain oil, black pepper oil, frankincense oil, vetivert oil, peppermint oil, bergamot oil, benzoin oil, aniba rosaeodora oil, marjoram oil, mandarin oil, myrrh oil, melissa oil, eucalyptus oil, Chinese lemon oil, lime oil, ravensara oil, lavandin oil, lavender oil, lindane oil, lemon oil, lemongrass oil, rose oil, rosewood oil, rosemary oil and lovage oil;

terpenes such as limonene, pinene, terpinene, terpinolene, myrcene and longifolene;

perfumes and water.

### <Emulsified Cosmetic>

The use of the emulsified skin external preparation is not particularly limited. As an example, the preparation may be suitably used as emulsified cosmetics that are the most frequent form of emulsified skin external preparations. The emulsified cosmetics are not particularly limited as long as they are used directly on skin. The emulsified skin external preparation has a wide range of uses including milky lotions, sera, creams and gels, and may be used regardless of user's gender and age.

The emulsified cosmetic according to the present invention may contain existing cosmetic ingredients while still achieving the effects of the invention.

The existing cosmetic ingredients include those listed in the following documents: The Japanese Standards of Cosmetic Ingredients 2nd edition (edited by Society of Japanese Pharmacopoeia and published by Yakuji Nippo, Ltd. (1984)), The Japanese Cosmetic Ingredients Codex (edited by Ministry of Health and Welfare, Pharmaceutical Examination Division and published by Yakuji Nippo, Ltd. (1993)), Supplement to The Japanese Cosmetic Ingredients Codex (edited by Ministry of Health and Welfare, Pharmaceutical Examination Division and published by Yakuji Nippo, Ltd. (1993)), The Comprehensive Licensing Standards of Cosmetics by Category (edited by Ministry of Health and Welfare, Pharmaceutical Examination Division and published by Yakuji Nippo, Ltd. (1993)), The Japanese Cosmetic Ingredients Codex by Category (edited by Ministry of Health and Welfare, Pharmaceutical Examination Division and published by Yakuji Nippo, Ltd. (1997)), and Dictionary of Cosmetic Ingredients (Nikko Chemicals., Co. Ltd. (1991)).

The emulsified skin external preparation and cosmetic of the present invention may be produced by common methods depending on the formulations, for example by dissolving, mixing or dispersing the aforesaid ingredients in predetermined amounts.

### <Method of Stabilizing Skin External Preparation Containing Salt of Ascorbic Acid Ester>

The skin external preparation of the invention contains the salt of ascorbic acid ester (A) in combination with the glycerin mono fatty acid ester (B), whereby the decomposition and consequent decrease of the salt of ascorbic acid ester (A) are prevented.

Specifically, the combined use of the salt of ascorbic acid ester (A) with the glycerin mono fatty acid ester (B) stabilizes the skin external preparation containing the salt of ascorbic acid ester (A). This method of stabilizing the skin external preparation containing the salt of ascorbic acid ester (A) is an aspect of the present invention.

### EXAMPLES

Hereinbelow, the present invention will be described in greater detail by examples. However, it should be construed that the invention is not limited thereto. The glycerin used in Examples had a purity of at least 98% by mass. In Examples and Comparative Examples, the storage stability was tested as follows.

### <Storage Stability Test>

The sample was allowed to stand in a thermostatic chamber at 40° C. for a month, and storage stability was evaluated by the remaining percentage (%) of the salt of ascorbic acid ester (A). The remaining percentage (%) was determined by the following formula:

Remaining percentage (%)=100×[concentration of salt(A) after storage at 40° C. for one month (%)/concentration of salt(A) immediately after preparation was produced (%)].

The concentration of the salt (A) was determined by high performance liquid chromatography under the following conditions:

### (Conditions of High Performance Liquid Chromatography)

Column: Shodex® C18P 4E (manufactured by SHOWA DENKO K.K.)

Column temperature: 40° C.

Eluting solution: 0.1 M dipotassium hydrogen phosphate (pH 7.0)/tetrahydrofuran=65/35

Flow rate: 0.7 ml/min

Detection: UV 265 nm

### Examples 1-7 and Comparative Examples 1-2

The materials of component I were mixed together in the amounts shown in Table 1 at 80° C. and were dissolved. Separately, the materials of component II were mixed together in the amounts shown in Table 1 at 80° C. and were dissolved. The component II was added to the component I. The mixture was cooled with stirring and was emulsified. The emulsion was cooled to 30° C. to give a cream. The cream was tested for storage stability. The results are shown in Table 1, in which the values are % by mass except for the remaining percentage.

TABLE 1

| | | Ex. | | | | | | | Comp. Ex. | |
|---|---|---|---|---|---|---|---|---|---|---|
| | | 1 | 2 | 3 | 4 | 5 | 6 | 7 | 1 | 2 |
| I | Glyceryl stearate | 0.1 | 0.5 | 1.0 | 2.0 | 5.0 | 10.0 | 20.0 | | |
| | Stearic acid | | | | | | | | | 3.0 |
| | Hydrogenated rapeseed oil alcohol | 4.2 | 4.2 | 4.2 | 4.2 | 4.2 | 4.2 | 4.2 | 4.2 | 4.2 |
| | Isononyl isononanoate | 6.0 | 6.0 | 6.0 | 6.0 | 6.0 | 6.0 | 6.0 | 6.0 | 6.0 |
| | Squalane | 9.6 | 9.6 | 9.6 | 9.6 | 9.6 | 9.6 | 9.6 | 9.6 | 9.6 |
| | Octyldodecyl myristate | 4.8 | 4.8 | 4.8 | 4.8 | 4.8 | 4.8 | 4.8 | 4.8 | 4.8 |
| | POE (20) sorbitan stearate | 1.0 | 1.0 | 1.0 | 1.0 | 1.0 | 1.0 | 1.0 | 1.0 | 1.0 |
| | Tocopherol | 0.2 | 0.2 | 0.2 | 0.2 | 0.2 | 0.2 | 0.2 | 0.2 | 0.2 |
| | Propylparaben | 0.1 | 0.1 | 0.1 | 0.1 | 0.1 | 0.1 | 0.1 | 0.1 | 0.1 |
| | Xanthan gum | 0.1 | 0.1 | 0.1 | 0.1 | 0.1 | 0.1 | 0.1 | 0.1 | 0.1 |
| II | Sodium ascorbic acid-2-phosphoric | 1.0 | 1.0 | 1.0 | 1.0 | 1.0 | 1.0 | 1.0 | 1.0 | 1.0 |
| | acid-6-palmitate | | | | | | | | | |
| | 1,3-BG | 4.8 | 4.8 | 4.8 | 4.8 | 4.8 | 4.8 | 4.8 | 4.8 | 4.8 |
| | Glycerin | 4.8 | 4.8 | 4.8 | 4.8 | 4.8 | 4.8 | 4.8 | 4.8 | 4.8 |
| | Trisodium citrate | 0.4 | 0.4 | 0.4 | 0.4 | 0.4 | 0.4 | 0.4 | 0.4 | 0.4 |
| | Potassium hydroxide | | | | | | | | | 0.2 |
| | Purified water | 62.9 | 62.5 | 62.0 | 61.0 | 58.0 | 53.0 | 43.0 | 63.0 | 59.8 |

| | | | | | | | | | |
|---|---|---|---|---|---|---|---|---|---|
| Storage stability test | 98 | 99 | 100 | 100 | 100 | 99 | 98 | 87 | 76 |
| [Remaining percentage (%)] | | | | | | | | | |

As shown in Table 1, Examples 1 to 7 used glyceryl stearate, whilst Comparative Example 1 did not use glyceryl stearate and Comparative Example 2 replaced glyceryl stearate by stearic acid and potassium hydroxide. The creams of Examples 1 to 7 showed that the decrease of sodium ascorbic acid-2-phosphoric acid-6-palmitate was small, proving excellent storage stability.

### Examples 8-15

Creams were prepared as described above using the materials in the amounts shown in Table 2. The creams were tested for storage stability. The results are shown in Table 2, in which the values are % by mass except for the remaining percentage.

TABLE 2

| | | Ex. | | | | | | | |
|---|---|---|---|---|---|---|---|---|---|
| | | 8 | 9 | 10 | 11 | 12 | 13 | 14 | 15 |
| I | Glyceryl stearate | 3.0 | 3.0 | 3.0 | 3.0 | 3.0 | 3.0 | 3.0 | 3.0 |
| | Hydrogenated rapeseed oil alcohol | 6.3 | 6.3 | 6.3 | 6.3 | 6.3 | 6.3 | 6.3 | 6.3 |
| | Isononyl isononanoate | 3.0 | 3.0 | 3.0 | 3.0 | 3.0 | 3.0 | 3.0 | 3.0 |
| | Squalane | 4.8 | 4.8 | 4.8 | 4.8 | 4.8 | 4.8 | 4.8 | 4.8 |
| | Octyldodecyl myristate | 9.6 | 9.6 | 9.6 | 9.6 | 9.6 | 9.6 | 9.6 | 9.6 |
| | POE (20) sorbitan stearate | 4.0 | 4.0 | 4.0 | 4.0 | 4.0 | 4.0 | 4.0 | 4.0 |
| | Tocopherol acetate | 0.1 | 0.1 | 0.1 | 0.1 | 0.1 | 0.1 | 0.1 | 0.1 |
| | Methylparaben | 0.1 | 0.1 | 0.1 | 0.1 | 0.1 | 0.1 | 0.1 | 0.1 |
| | Hydroxyethyl cellulose | 0.1 | 0.1 | 0.1 | 0.1 | 0.1 | 0.1 | 0.1 | 0.1 |
| II | Sodium ascorbic acid-2-phosphoric | 0.05 | 0.1 | 0.5 | 1.0 | 2.0 | 5.0 | 10.0 | 20.0 |
| | acid-6-palmitate | | | | | | | | |
| | 1,3-BG | 2.4 | 2.4 | 2.4 | 2.4 | 2.4 | 2.4 | 2.4 | 2.4 |
| | Glycerin | 6.0 | 6.0 | 6.0 | 6.0 | 6.0 | 6.0 | 6.0 | 6.0 |
| | Trisodium citrate | 0.1 | 0.1 | 0.2 | 0.4 | 0.6 | 0.6 | 0.6 | 0.6 |
| | Purified water | 60.5 | 60.4 | 59.9 | 59.2 | 58.0 | 55.0 | 50.0 | 40.0 |

| | | | | | | | | |
|---|---|---|---|---|---|---|---|---|
| Storage stability test | 98 | 99 | 100 | 100 | 100 | 100 | 99 | 98 |
| [Remaining percentage (%)] | | | | | | | | |

As shown in Table 2, the creams of Examples 8 to 15 showed that the decrease of sodium ascorbic acid-2-phosphoric acid-6-palmitate was small, proving excellent storage stability.

### Examples 16-24

Creams were prepared as described above using the materials in the amounts shown in Table 3. The creams were tested for storage stability. The results are shown in Table 3, in which the values are % by mass except for the remaining percentage.

TABLE 3

| | | Ex. | | | | | | | | |
|---|---|---|---|---|---|---|---|---|---|---|
| | | 16 | 17 | 18 | 19 | 20 | 21 | 22 | 23 | 24 |
| I | Glyceryl laurate | | | 1.0 | 1.0 | | | | | |
| | Glyceryl myristate | 1.0 | | | | 1.0 | 1.0 | 1.0 | | |
| | Glyceryl stearate | | 1.0 | 1.0 | | 1.0 | | | 1.0 | 1.0 |
| | Glyceryl isostearate | | | | 1.0 | | 1.0 | | | |
| | Glyceryl oleate | | | | | | | 1.0 | 1.0 | |
| | Glyceryl ricinoleate | | | | | | | | | 1.0 |
| | Hydrogenated rapeseed oil alcohol | 4.2 | 4.2 | 4.2 | 4.2 | 4.2 | 4.2 | 4.2 | 4.2 | 4.2 |
| | Isononyl isononanoate | 6.0 | 6.0 | 6.0 | 6.0 | 6.0 | 6.0 | 6.0 | 6.0 | 6.0 |
| | Squalane | 9.6 | 9.6 | 9.6 | 9.6 | 9.6 | 9.6 | 9.6 | 9.6 | 9.6 |
| | Octyldodecyl myristate | 4.8 | 4.8 | 4.8 | 4.8 | 4.8 | 4.8 | 4.8 | 4.8 | 4.8 |
| | Polyglyceryl (10) monostearate | 2.0 | 2.0 | 2.0 | 2.0 | 2.0 | 2.0 | 2.0 | 2.0 | 2.0 |
| | Tocopherol | 0.2 | 0.2 | 0.2 | 0.2 | 0.2 | 0.2 | 0.2 | 0.2 | 0.2 |
| | Propylparaben | 0.1 | 0.1 | 0.1 | 0.1 | 0.1 | 0.1 | 0.1 | 0.1 | 0.1 |
| | Xanthan gum | 0.1 | 0.1 | 0.1 | 0.1 | 0.1 | 0.1 | 0.1 | 0.1 | 0.1 |
| II | Sodium ascorbic acid-2-phosphoric | 1.0 | 1.0 | 1.0 | 1.0 | 1.0 | 1.0 | 1.0 | 1.0 | 1.0 |
| | acid-6-palmitate | | | | | | | | | |
| | 1,3-BG | 4.8 | 4.8 | 4.8 | 4.8 | 4.8 | 4.8 | 4.8 | 4.8 | 4.8 |
| | Glycerin | 4.8 | 4.8 | 4.8 | 4.8 | 4.8 | 4.8 | 4.8 | 4.8 | 4.8 |
| | Trisodium citrate | 0.4 | 0.4 | 0.4 | 0.4 | 0.4 | 0.4 | 0.4 | 0.4 | 0.4 |
| | Purified water | 61.0 | 61.0 | 60.0 | 60.0 | 60.0 | 60.0 | 60.0 | 60.0 | 60.0 |

| | | | | | | | | | |
|---|---|---|---|---|---|---|---|---|---|
| Storage stability test | 100 | 100 | 100 | 100 | 100 | 100 | 100 | 100 | 100 |
| [Remaining percentage (%)] | | | | | | | | | |

As shown in Table 3, the creams of Examples 16 to 24 showed that the decrease of sodium ascorbic acid-2-phosphoric acid-6-palmitate was small, proving excellent storage stability.

### Example 25

A cream was prepared as described above using the materials in the amounts shown in Table 4. The cream was tested for storage stability. The result is shown in Table 4, in which the values are % by mass except for the remaining percentage.

TABLE 4

| | | Ex. 25 |
|---|---|---|
| I | Glyceryl stearate | 1.0 |
| | Stearic acid | 1.2 |
| | Cetostearyl alcohol | 1.2 |
| | Sorbitan sesquioleate | 1.2 |
| | Tea tree oil | 0.1 |
| | Synthetic sodium magnesium silicate | 0.4 |
| | Dipotassium glycyrrhizinate | 0.1 |
| | Dicapryl carbonate | 2.4 |
| | Olive oil | 4.8 |
| | Octyldodecyl neopentanoate | 4.8 |
| | Tocopherol | 0.1 |
| | Jojoba oil | 4.8 |
| | 2-Octyldodecanol | 2.4 |
| | Polysorbate 60 | 4.8 |
| | 1,2-Hexanediol | 2.4 |
| | Glycerin | 4.8 |
| | Polyquaternium 51 | 0.3 |
| | Polyvinylpyrrolidone | 0.1 |
| | Acrylate/alkyl acrylate (C10-30) | 0.2 |
| | cross polymer | |
| II | Sodium ascorbic acid-2-phosphoric | 1.0 |
| | acid-6-palmitate | |
| | Sodium ascorbic acid-2-phosphoric | 1.0 |
| | acid-6-(2-hexyldecanoate) | |
| | Sodium lactate | 0.4 |
| | Sodium tartrate | 0.4 |
| | Sodium pyrrolidonecarboxylate | 0.2 |
| | Arginine | 0.1 |
| | Serine | 0.1 |
| | Tetrasodium hydroxyethane | 0.4 |
| | diphosphonate | |
| | Purified water | 59.3 |

| | |
|---|---|
| Storage stability test | 100 |
| [Remaining percentage (%)] | |

As shown in Table 4, the cream of Example 25 showed that the decrease of sodium ascorbic acid-2-phosphoric acid-6-palmitate and sodium ascorbic acid-2-phosphoric acid-6-(2-hexyldecanoate) was small, proving excellent storage stability.

### Example 26

A cream was prepared as described above using the materials in the amounts shown in Table 5. The cream was tested for storage stability. The result is shown in Table 5, in which the values are % by mass except for the remaining percentage.

TABLE 5

| | | Ex. 26 |
|---|---|---|
| I | Glyceryl stearate | 2.0 |
| | Cyclomethicone | 1.0 |
| | Squalane | 7.0 |
| | Isononyl isononanoate | 5.0 |
| | Glyceryl tri(caprylate/caprate) | 2.0 |
| | Cetyl palmitate | 1.0 |
| | Cholesterol | 0.5 |
| | Hydrogenated lecithin | 0.5 |
| | Polyglyceryl-10 myristate | 2.0 |
| | POE (60) hydrogenated castor oil | 0.4 |
| | Cetanol | 2.0 |
| | Stearyl alcohol | 1.0 |
| | Glyceryl 2-ethylhexanoate | 4.0 |
| | PEG-60 stearate | 1.0 |
| | Tocopherol acetate | 0.8 |
| | Phenoxyethanol | 0.4 |
| | Propylparaben | 0.1 |
| II | Sodium ascorbic acid-2-phosphoric | 1.0 |
| | acid-6-palmitate | |
| | Dipotassium glycyrrhizinate | 0.2 |
| | Carbomer | 0.1 |
| | Diglycerin | 3.0 |
| | Pentylene glycol | 2.0 |
| | Methylparaben | 0.1 |
| | Citric acid | 0.1 |
| | Trisodium citrate | 1.0 |
| | Tetrasodium EDTA | 0.2 |
| | Magnesium sulfate | 1.0 |
| | Potassium hydroxide | 0.1 |
| | Purified water | 60.5 |

| | |
|---|---|
| Storage stability test | 100 |
| [Remaining percentage (%)] | |

As shown in Table 5, the cream of Example 26 showed that the decrease of sodium ascorbic acid-2-phosphoric acid-6-palmitate was small, proving excellent storage stability.

1. An emulsified skin external preparation comprising 0.03 to 25% by mass of a salt of a higher fatty acid ester of ascorbic acid-2-phosphate (A), and 0.05 to 25% by mass of a glycerin mono fatty acid ester (B), the higher fatty acid ester of ascorbic acid-2-phosphate being represented by Formula (1): 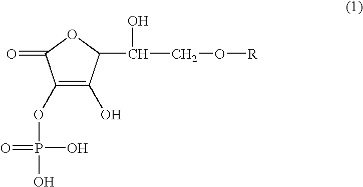
wherein R is a higher fatty acid residue.

2. The emulsified skin external preparation according to claim 1, wherein R in Formula (1) is a residue of an aliphatic carboxylic acid of 10 to 20 carbon atoms.

3. The emulsified skin external preparation according to claim 2, wherein the aliphatic carboxylic acid of 10 to 20 carbon atoms is lauric acid, myristic acid, palmitic acid, stearic acid, 2-hexyldecanoic acid or isostearic acid.

4. The emulsified skin external preparation according to claim 2, wherein the aliphatic carboxylic acid of 10 to 20 carbon atoms is palmitic acid.

5. The emulsified skin external preparation according to claim 2, wherein the aliphatic carboxylic acid of 10 to 20 carbon atoms is 2-hexyldecanoic acid.

6. The emulsified skin external preparation according to any one of claims 1 to 5, wherein the component (A) is a sodium salt.

7. The emulsified skin external preparation according to claim 1, wherein the glycerin mono fatty acid ester (B) is at least one ester selected from the group consisting of glyceryl caprylate, glyceryl caprate, glyceryl laurate, glyceryl myristate, glyceryl stearate, glyceryl isostearate, glyceryl behenate, glyceryl linoleate, glyceryl oleate, glyceryl cocofatty acid ester, glyceryl ricinoleate, glyceryl hydroxystearate and glyceryl erucate.

8. An emulsified cosmetic comprising the emulsified skin external preparation claimed in any one of claims 1 to 7.

9. A method for stabilizing an emulsified skin external preparation, characterized in that a salt of a higher fatty acid ester of ascorbic acid-2-phosphate (A) is used in combination with a glycerin mono fatty acid ester (B), the higher fatty acid ester of ascorbic acid-2-phosphate being represented by Formula (1): 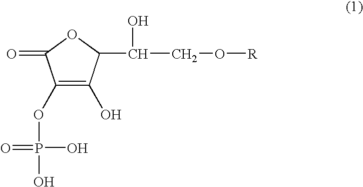
wherein R is a higher fatty acid residue.
